(19) United States
(12) Patent Application Publication
(10) Pub. No.: US 2024/0130614 A1
(43) Pub. Date: Apr. 25, 2024

(54) INTRAOCULAR PRESSURE INSPECTION DEVICE

(71) Applicant: Medimaging Integrated Solution, Inc., Hsinchu (TW)

(72) Inventors: Shao Hung HUANG, Hsinchu (TW); Chao-Ting CHEN, Hsinchu (TW); Fong Hao KUO, Hsinchu (TW); Yu-Chung TUNG, Hsinchu (TW); Chu-Ming CHENG, Hsinchu (TW); Chi-Yuan KANG, Hsinchu (TW)

(21) Appl. No.: 18/486,676

(22) Filed: Oct. 13, 2023

(30) Foreign Application Priority Data
Oct. 14, 2022 (TW) 111139150

(51) Int. Cl. A61B 3/16

(52) U.S. Cl. CPC A61B 3/165

(57) ABSTRACT

An intraocular pressure inspection device includes an intraocular pressure detection unit, a high-precision positioning system and a wide-area positioning system, wherein according to the position of the intraocular pressure detection unit, a set of high-precision coordinates output by the high-precision positioning system and a set of wide-area coordinates output by the wide-area positioning system are integrated in appropriate weights to obtain a set of more precise integrated coordinate. The above-mentioned intraocular pressure inspection device can prevent the intraocular pressure detection unit from failing to operate once it is not in the working area of the high-precision positioning system.

### BACKGROUND OF THE INVENTION

### 1. Field of the Invention

The present invention relates to an intraocular pressure inspection device, particularly to an intraocular pressure inspection device able to automatically align itself to the vertex of the curved surface of the eyeball.

### 2. Description of the Prior Art

An eyeball must maintain intraocular pressure (IOP) within a given range to support the elasticity and visual function thereof. However, too high intraocular pressure would compress nerves, damage nervous function, induce visual field defect, degrade eyesight, and finally lead to glaucoma.

The frequently-used intraocular pressure inspection devices include the applanation tonometer and the pneumatic tonometer. In the applanation tonometry, an anesthetic is dripped on the cornea beforehand, and then the tonometer is moved to contact the cornea for measuring intraocular pressure. Therefore, it is more difficult to operate the applanation tonometer. In the pneumatic tonometry, a blast of air is injected to the vertex of the curved surface of the eyeball at a given pressure to deform the surface of the eyeball, and then the surface deformation of the eyeball is measured to calculate the intraocular pressure. Therefore, the pneumatic tonometer can measure intraocular pressure in a non-contact way.

In order to detect the vertex of the curved surface of the eyeball, the conventional pneumatic tonometer is equipped with a high-precision positioning system. The high-precision positioning system outputs the coordinates of the vertex of the curved surface of the eyeball. According to the coordinates, the operator moves the intraocular detection unit and aligns it to the vertex of the curved surface of the eyeball. Then, the operator injects air to the vertex of the curved surface of the eyeball and measures the intraocular pressure. However, the high-precision positioning system can only work in a very narrow area. Only well-trained operators can obtain accurate measurement results. Thus, the testees must go to hospital for tonometry. Such an inconvenience will make the testees decrease the frequency of tonometry.

The numerical-control triaxial servo table can align the intraocular pressure detection unit to the vertex of the curved surface of the eyeball according to the coordinates output by the high-precision positioning system. However, the control of the triaxial servo table is likely to depart from the working area of the high-precision positioning system because of too narrow a working area of the high-precision positioning system, system noise, and the responsive characteristics of the motor. Thus, the system may fail to work normally.

Accordingly, the concerned manufacturers are eager to development an intraocular pressure inspection device able to automatically align itself to the vertex of the curved surface of the eyeball.

### SUMMARY OF THE INVENTION

The present invention provides an intraocular pressure inspection device, which comprises a high-precision positioning system and a wide-area positioning system, and which integrates a set of high-precision coordinates output by the high-precision positioning system and a set of wide-area coordinates output by the wide-area positioning system in appropriate weights to obtain a set of more precise integrated coordinates to overcome the problems occurring in the situation that the control of the triaxial servo table departs from the working area of the high-precision positioning system.

In one embodiment, the intraocular pressure inspection device of the present invention comprises an intraocular pressure detection unit, a high-precision positioning system, a wide-area positioning system, a triaxial servo table, and a processor. The intraocular pressure detection unit injects air to an eyeball and measures the intraocular pressure of the eyeball. The high-precision positioning system measures a target position of the eyeball and outputs a set of high-precision coordinates. The wide-area positioning system measures the target position and outputs a set of wide-area coordinates. The triaxial servo table is coupled to the intraocular pressure detection unit and moves the intraocular pressure detection unit. The processor is electrically connected with the high-precision positioning system, the wide-area positioning system, and the triaxial servo table. According to a reference distance between the intraocular detection unit and the vertex of the curved surface of the eyeball, the processor adjusts the weights of the high-precision coordinates and the wide-area coordinates to work out a set of integrated coordinates and controls the triaxial servo table to move the intraocular detection unit to the integrated coordinates.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a block diagram schematically showing the system of an intraocular pressure inspection device according to one embodiment of the present invention;

FIG. 2 is a diagram schematically showing the structure of an intraocular pressure inspection device according to one embodiment of the present invention;

FIG. 3 is a diagram showing the relationship of a reference distance and a proportional control parameter according to one embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

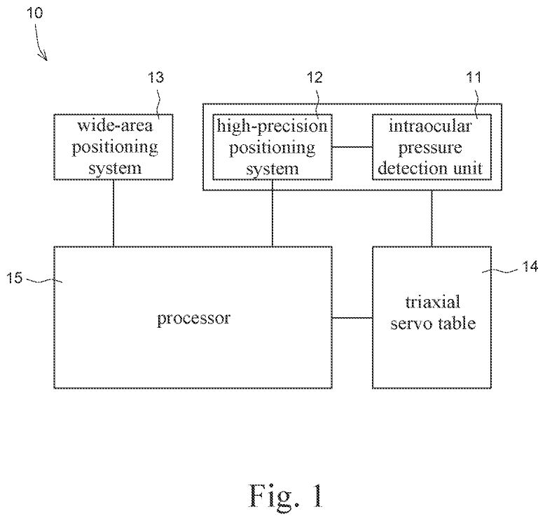

Refer to FIG. 1. In one embodiment, the intraocular pressure inspection device 10 of the present invention comprises an intraocular pressure detection unit 11, a high-precision positioning system 12, a wide-area positioning system 13, a triaxial servo table 14, and a processor 15. The intraocular pressure detection unit 11 measures the intraocular pressure of an eyeball. For example, the intraocular pressure detection unit 11 may be a pneumatic tonometer, which injects air to the vertex of the curved surface of an eyeball to make the surface of the eyeball deform and then measure the magnitude of the deformation to work out the intraocular pressure. The main components of the intraocular pressure detection unit 11, such as the air-injection module, the light emitter, and the light receiver, are familiarized with by the persons having ordinary knowledge in the field and will not repeat herein. The high-precision positioning system 12 measures a target position, such as the vertex of the curved surface of the eyeball, and outputs a set of high-precision coordinates. The wide-area positioning system 13 measures the target position and outputs a set of wide-area coordinates.

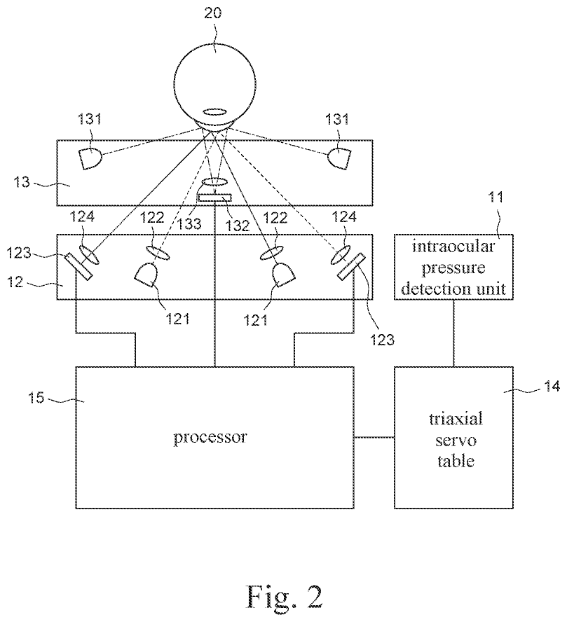

Refer to FIG. 2. In one embodiment, the high-precision positioning system 12 includes a first light source 121 and a light sensor 123. The first light source 121 generates a collimated light to illuminate the curved surface of the eyeball 20. In one embodiment, a lens 122 is disposed on the light output side of the first light source 121 to collimate the light generated by the first light source 121. The light sensor 123 receives the collimated light reflected by the curved surface of the eyeball 20 and works out the position of the vertex of the curved surface of the eyeball 20, i.e. the high-precision coordinates. In one embodiment, a lens 124 is disposed on the light input side of the light sensor 123.

Refer to FIG. 2. In one embodiment, the wide-area positioning system 13 includes a second light source 131 and an image sensor 132. The second light source 131 generates a structured light and projects the structured light onto the curved surface of the eyeball 20. The image sensor 132 captures an image generated by the projected structured light. In one embodiment, a lens 133 is disposed on the light input side of the image sensor 132. The magnitude of the deformation of the structured light may be used to estimate the position of the vertex of the curved surface of the eyeball 20, i.e. the wide-area coordinates.

The triaxial servo table 14 is coupled to the intraocular pressure detection unit 11. The triaxial servo table 14 moves the intraocular pressure detection unit 11 to align the intraocular pressure detection unit 11 to the vertex of the curved surface of the eyeball 20 for intraocular pressure measurement. The processor 15 is electrically connected with the high-precision positioning system 12, the wide-area positioning system 13, and the triaxial servo table 14. According to a reference distance between the intraocular pressure detection unit 11 and the vertex of the curved surface of the eyeball 20, the processor 15 adjusts the weights of the high-precision coordinates output by the high-precision positioning system 12 and the wide-area coordinates output by the wide-area positioning system 13 to work out a set of integrated coordinates. According to the integrated coordinates, the processor 15 controls the triaxial servo table 14 to move the intraocular pressure detection unit 11 to the integrated coordinates. The user may refer to the integrated coordinates for the position of the vertex of the curved surface of the eyeball 20. It is easily understood: while not in the working area of the high-precision positioning system 12, the control of the triaxial servo table relies on the wide-area coordinates output by the wide-area positioning system 13. Therefore, the integrated coordinates worked out by the processor 15 may be deviated from the real vertex of the curved surface of the eyeball 20. However, after the abovementioned step has been performed for a plurality of times, the intraocular pressure detection unit 11 will approach the vertex of the curved surface of the eyeball 20 and enter the working area of the high-precision positioning system 12.

The method for calculating the integrated coordinates is introduced below. The integrated coordinates may be worked out from Equation (1):

$$[ \begin{matrix}\overline{x} \\ \overline{y} \\ \overline{z}\end{matrix} ] = r [ \begin{matrix}x \\ y \\ z\end{matrix} ] + ( 1 - r ) ( [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] [ \begin{matrix}X \\ Y \\ Z\end{matrix} ] + [ \begin{matrix}{b}_{1} \\ {b}_{2} \\ {b}_{3}\end{matrix} ] ) \qquad ( 1 )$$

wherein x̅, y̅ and z̅ are a set of integrated coordinates; x, y and z are a set of high-precision coordinates output by the high-precision positioning system 12; X, Y and Z are a set of wide-area coordinates output by the wide-area positioning system 13; r is the weight; a and b are the coefficients. In one embodiment, while the reference distance is smaller than a first preset value, the weight r is 1, i.e. the set of integrated coordinates equal the set of high-precision coordinates output by the high-precision positioning system 12; while the reference distance is greater than or equal to a second preset value, the weight r is 0, i.e. the set of integrated coordinates equal the set of wide-area coordinates output by the wide-area positioning system 13; while the reference distance is greater than or equal to the first preset value and smaller than the second preset value, the weight r is worked out from Equation (2):

$$r = \frac{{t}_{2} - d}{( {t}_{2} - {t}_{1} )} \qquad ( 2 )$$

wherein r is the weight; d is the reference distance; t₁ is the first preset value; t₂ is the second preset value. According to Equations (1) and (2), while the reference distance is greater than or equal to the first preset value and smaller than the second preset value, the set of integrated coordinates can more stably vary between a set of high-precision coordinates output by the high-precision positioning system 12 and a set of wide-area coordinates output by the wide-area positioning system 13. It is easily understood: the weight is not necessarily adjusted according to the abovementioned method but may be otherwise adjusted according to different requirements.

The coefficients a and b in Equation (1) may be defined by the user himself according to practical design. In one embodiment, the coefficients a and b make Equation (3) established and make Equation (4) have a minimum value.

$$[ \begin{matrix}\hat{x} \\ \hat{y} \\ \hat{z}\end{matrix} ] = [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] [ \begin{matrix}{X}_{i} \\ {Y}_{i} \\ {Z}_{i}\end{matrix} ] + [ \begin{matrix}{b}_{1} \\ {b}_{2} \\ {b}_{3}\end{matrix} ] ) \qquad ( 3 )$$

$${\sum }_{i} {( {\hat{x}}_{i} - {x}_{i} )}^{2} + {( {\hat{y}}_{i} - {y}_{i} )}^{2} + {( {\hat{z}}_{i} - {z}_{i} )}^{2} \qquad ( 4 )$$

wherein Xᵢ, Yᵢ and Zᵢ are a set of wide-area coordinates of Point i inside the working area of the high-precision positioning system 12; xᵢ, yᵢ and zᵢ are a set of high-precision coordinates of Point i ; \hat{x}ᵢ, ŷᵢ and \hat{z}ᵢ are a set of calibration coordinates of Point i. In one embodiment, the coefficients a and b may be obtained via using a smooth curved surface having a curvature similar to that of the surface of the eyeball to calibrate the intraocular pressure inspection device 10. For example, the user may select n positions for Point i within the working area of the high-precision positioning system 12 for calibration, wherein n≥4, and i=1,2, . . . n. Moving the intraocular pressure detection unit to Point i for positioning measurement may obtain a set of high-precision coordinates xᵢ, yᵢ and zᵢ output by the high-precision positioning system 12 and a set of wide-area coordinates Xᵢ, Yᵢ and Zᵢ output by the wide-area positioning system 13 and establish Equation (5):

$$[ \begin{matrix}{X}_{1} & {Y}_{1} & {Z}_{1} & 0 & 0 & 0 & 0 & 0 & 0 & 1 & 0 & 0 \\ 0 & 0 & 0 & {X}_{1} & {Y}_{1} & {Z}_{1} & 0 & 0 & 0 & 0 & 1 & 0 \\ 0 & 0 & 0 & 0 & 0 & 0 & {X}_{1} & {Y}_{1} & {Z}_{1} & 0 & 0 & 1 \\ {X}_{2} & {Y}_{2} & {Z}_{2} & 0 & 0 & 0 & 0 & 0 & 0 & 1 & 0 & 0 \\ 0 & 0 & 0 & {X}_{2} & {Y}_{2} & {Z}_{2} & 0 & 0 & 0 & 0 & 1 & 0 \\ 0 & 0 & 0 & 0 & 0 & 0 & {X}_{2} & {Y}_{2} & {Z}_{2} & 0 & 0 & 1 \\ & ⋱ & & & ⋱ & & & ⋱ & & & ⋱ & \\ {X}_{n} & {Y}_{n} & {Z}_{n} & 0 & 0 & 0 & 0 & 0 & 0 & 1 & 0 & 0 \\ 0 & 0 & 0 & {X}_{n} & {Y}_{n} & {Z}_{n} & 0 & 0 & 0 & 0 & 1 & 0 \\ 0 & 0 & 0 & 0 & 0 & 0 & {X}_{n} & {Y}_{n} & {Z}_{n} & 0 & 0 & 1\end{matrix} ] [ \begin{matrix}{a}_{11} \\ {a}_{12} \\ {a}_{13} \\ {a}_{21} \\ {a}_{22} \\ {a}_{23} \\ {a}_{31} \\ {a}_{32} \\ {a}_{33} \\ {b}_{1} \\ {b}_{2} \\ {b}_{3}\end{matrix} ] = [ \begin{matrix}{x}_{1} \\ {y}_{1} \\ {z}_{1} \\ {x}_{2} \\ {y}_{2} \\ {z}_{2} \\ ⋮ \\ {x}_{n} \\ {y}_{n} \\ {z}_{n}\end{matrix} ] \qquad ( 5 )$$

A linear algebraic method may be used to obtain the solution of the coefficients a and b.

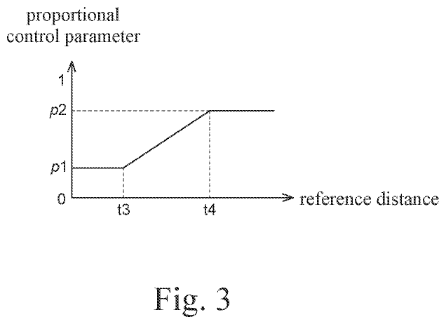

In one embodiment, the processor 15 uses a PID (Proportional-Integral-Derivative) controller to control the movement of the triaxial servo table 14. For example, the processor 15 controls the movement of the triaxial servo table 14 with Equation (6):

$$[ \begin{matrix}{s}_{x} \\ {s}_{y} \\ {s}_{z}\end{matrix} ] = pM [ \begin{matrix}\overline{x} \\ \overline{y} \\ \overline{z}\end{matrix} ] \qquad ( 6 )$$

wherein sₓ, s_{y} and s_{z} are the advancing steps of the triaxial servo table 14; x̅, y̅ and z̅ are a set of integrated coordinates worked out by the processor 15; M is a geometrical transformation matrix, which is determined by the lengths of the advancing steps, the magnification ratio of the integrated coordinates, and the relative rotation; p is a proportional control parameter of the PID controller. In one embodiment, the proportional control parameter is defined using a fuzzy logic method. Refer to FIG. 3. The horizontal axis is a reference distance from the intraocular pressure detection unit 11 to the target position of the eyeball 20 (such as the vertex of the curved surface). The vertical axis is the proportional control parameter p. While the reference distance is smaller than a third preset value t3, the proportional control parameter p is a smaller proportional control parameter p1. In other words, the triaxial servo table 14 has fewer advancing steps. While the reference distance is greater than or equal to a fourth preset value t4, the proportional control parameter p is a greater proportional control parameter p2. In other words, the triaxial servo table 14 has more advancing steps. While the reference distance is greater than or equal to the third preset value and smaller than the fourth preset value, the proportional control parameter p varies between the proportional control parameter p1 and the proportional control parameter p2. It is easily understood: the proportional control parameter p may be appropriately modified according to the practically required control method.

It should be understood: the intraocular pressure detection unit 11 cannot measure the intraocular pressure unless the intraocular pressure detection unit 11 is aligned to the vertex of the curved surface of the eyeball 20. The high-precision positioning system 12 will obtain more precise positioning coordinates within the working area thereof. In one embodiment, the high-precision positioning system 12 is electrically connected with the intraocular pressure detection unit 11; once the intraocular pressure detection unit 11 is exactly aligned to the vertex of the curved surface of the eyeball 20, the high-precision positioning system 12 triggers the intraocular pressure detection unit 11 to measure intraocular pressure. In one embodiment, the triaxial servo table 14 moves the high-precision positioning system 12 and the intraocular pressure detection unit 11 simultaneously to make the working area of the high-precision positioning system 12 approach and cover the vertex of the curved surface of the eyeball 20.

According to the abovementioned architecture, while the intraocular pressure detection unit 11 is deviated from the vertex of the curved surface of the eyeball 20 to a degree, the processor 15 may increase the weight of a set of wide-area coordinates to work out a set of integrated coordinates and control the triaxial servo table 14 to drive the intraocular pressure detection unit 11 to approach the vertex of the curved surface of the eyeball 20 faster. While the intraocular pressure detection unit 11 is close to the vertex of the curved surface of the eyeball 20, the processor 15 increases the weight of the set of high-precision coordinates to work out a set of integrated coordinates and controls the triaxial servo table 14 to drive the intraocular pressure detection unit 11 slower lest the intraocular pressure detection unit 11 be out of the working area of the high-precision positioning system 12. Therefore, the intraocular pressure inspection device of the present invention enables the intraocular pressure detection unit 11 to be aligned to the vertex of the curved surface of the eyeball 20 faster and more precisely. It can be learned from the above description: via using the integrated coordinates and using the triaxial servo table to move the intraocular pressure detection unit 11, the intraocular pressure detection unit 11 can be automatically aligned to the vertex of the curved surface of the eyeball 20 for measuring intraocular pressure. Therefore, the intraocular pressure inspection device of the present invention can be operated by the testee himself to measure intraocular pressure.

In conclusion, the present invention provides an intraocular pressure inspection device, which comprises a high-precision positioning system and a wide-area positioning system, wherein appropriate weights are used to integrate a set of high-precision coordinates output by the high-precision positioning system and a set of wide-area coordinates output by the wide-area positioning system to obtain a set of more precise integrated coordinates. Besides, the present invention uses a fuzzy logic method to control the triaxial servo table to move the intraocular pressure detection unit in different speeds lest the control of the triaxial servo table depart from the working area of the high-precision positioning system.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

What is claimed is:

1. An intraocular pressure inspection device, comprising
an intraocular pressure detection unit, injecting air to an eyeball and measuring intraocular pressure of the eyeball;
a high-precision positioning system, measuring a target position of the eyeball and outputting a set of high-precision coordinates;
a wide-area positioning system, measuring the target position and outputting a set of wide-area coordinates;
a triaxial servo table, coupled to the intraocular pressure detection unit and moving the intraocular pressure detection unit; and
a processor, electrically connected with the high-precision positioning system, the wide-area positioning system and the triaxial servo table, wherein according to a reference distance between the intraocular detection unit and a vertex of a curved surface of the eyeball, the processor adjusts weights of the set of high-precision coordinates and the set of wide-area coordinates to work out a set of integrated coordinates and controls the triaxial servo table to move the intraocular detection unit to the set of integrated coordinates.

2. The intraocular pressure inspection device according to claim 1, wherein the set of integrated coordinates are obtained via the following equation:
$$[ \begin{matrix}\overline{x} \\ \overline{y} \\ \overline{z}\end{matrix} ] = r [ \begin{matrix}x \\ y \\ z\end{matrix} ] + ( 1 - r ) ( [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] [ \begin{matrix}X \\ Y \\ Z\end{matrix} ] + [ \begin{matrix}{b}_{1} \\ {b}_{2} \\ {b}_{3}\end{matrix} ] )$$
wherein x̅, y̅ and z̅ are a set of integrated coordinates; x, y and z are a set of high-precision coordinates output by the high-precision positioning system; X, Y and Z are a set of wide-area coordinates output by the wide-area positioning system; r is a weight; a and b are coefficients.

3. The intraocular pressure inspection device according to claim 2, wherein the coefficients make established the following condition:
$$[ \begin{matrix}\hat{x} \\ \hat{y} \\ \hat{z}\end{matrix} ] = [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] [ \begin{matrix}{X}_{i} \\ {Y}_{i} \\ {Z}_{i}\end{matrix} ] + [ \begin{matrix}{b}_{1} \\ {b}_{2} \\ {b}_{3}\end{matrix} ] )$$
and make Σᵢ(\hat{x}ᵢ−xᵢ)²+(ŷᵢ−yᵢ)²+(\hat{z}ᵢ−zᵢ)² have a minimum value, wherein Xᵢ, Yᵢ and Zᵢ are a set of wide-area coordinates of Point i inside a working area of the high-precision positioning system; xᵢ, yᵢ and zᵢ are a set of high-precision coordinates of Point i; \hat{x}ᵢ, y̅ᵢ and z̅ᵢ are a set of calibration coordinates of Point i.

4. The intraocular pressure inspection device according to claim 2, wherein while the reference distance is greater than or equal to a first preset value and smaller than a second preset value, the weight is worked out according to the following equation:
$$r = \frac{{t}_{2} - d}{( {t}_{2} - {t}_{1} )}$$
wherein r is the weight; d is the reference distance; t₁ is the first preset value; t₂ is the second preset value.

5. The intraocular pressure inspection device according to claim 1, wherein while the reference distance is smaller than a first preset value, the set of integrated coordinates equal the set of high-precision coordinates.

6. The intraocular pressure inspection device according to claim 1, wherein while the reference distance is greater than or equal to a second preset value, the set of integrated coordinates equal the set of wide-area coordinates.

7. The intraocular pressure inspection device according to claim 1, wherein, the processor uses a PID (Proportional-Integral-Derivative) controller to control a movement of the triaxial servo table.

8. The intraocular pressure inspection device according to claim 1, wherein the processor controls a movement of the triaxial servo table with the following equation:
$$[ \begin{matrix}{s}_{x} \\ {s}_{y} \\ {s}_{z}\end{matrix} ] = pM [ \begin{matrix}\overline{x} \\ \overline{y} \\ \overline{z}\end{matrix} ]$$
wherein sₓ, s_{y} and s_{z} are the advancing steps of the triaxial servo table; x̅, y̅ and z̅ are a set of integrated coordinates; M is a geometrical transformation matrix; p is a proportional control parameter of the PID controller.

9. The intraocular pressure inspection device according to claim 8, wherein the proportional control parameter is defined using a fuzzy logic method.

10. The intraocular pressure inspection device according to claim 1, wherein the high-precision positioning system includes
at least one first light source, generating a collimated light to illuminate the eyeball; and
a light sensor, receiving the collimated light reflected by the eyeball for working out the set of high-precision coordinates.

11. The intraocular pressure inspection device according to claim 1, wherein the wide-area positioning system includes
a second light source, generating a structured light and projecting the structured light onto the eyeball; and
an image sensor, capturing an image of the eyeball generated by the structured light projected onto the eyeball for calculating the set of wide-area coordinates.

12. The intraocular pressure inspection device according to claim 1, wherein the high-precision positioning system is electrically connected with the intraocular pressure detection unit and triggers the intraocular pressure detection unit to measure intraocular pressure.

13. The intraocular pressure inspection device according to claim 1, wherein the triaxial servo table moves the high-precision positioning system and the intraocular pressure detection unit simultaneously.

14. The intraocular pressure inspection device according to claim 1, wherein the target position is the vertex of the curved surface of the eyeball.
